(19) United States
(12) Patent Application Publication
(10) Pub. No.: US 2022/0338815 A1
(43) Pub. Date: Oct. 27, 2022

(54) INFLATABLE HEAD SUPPORT

(71) Applicant: VICTORIA LINK LIMITED, Wellington (NZ)

(72) Inventors: Mailin LEMKE, Lower Hutt (NZ); Jacob MARSH, Wellington (NZ); Benjamin John PARKINSON, Lower Hutt (NZ); Edgar Rodriguez RAMIREZ, Lower Hutt (NZ)

(21) Appl. No.: 17/765,079

(22) PCT Filed: Oct. 2, 2020
(86) PCT No.: PCT/NZ2020/050117

§ 371 Date: Mar. 30, 2022

(30) Foreign Application Priority Data
Oct. 3, 2019 (AU) 2019903719

(51) Int. Cl. A61B 5/00; A61G 13/12

(52) U.S. Cl. CPC A61B 5/702; A61G 13/121; A61G 13/1265; A61G 2210/50; A61G 2200/34

(57) ABSTRACT

The invention relates to a stabilising head support for use by a patient during MRI scanning. The head support includes a shell and an inflatable bladder that is located at least partially within the shell. During use, the shell is located within an MRI magnet and the bladder is inflated about the patients head to help hold the head steady during imaging.

### BACKGROUND

### Field of the Invention

The present disclosure generally relates to an inflatable head support to be used by a patient during magnetic resonance imaging (MRI).

### Description of the Related Art

MRI is a technology used for capturing detailed images of different tissues within your body for diagnosing or examining many medical conditions. Currently, the infrastructure, portability, cost, and lack of movement in imaging are all components that limit the access to this technology. The development of a head-only MRI system with a reduced size, weight and associated costs could make the technology transportable and consequently, reach a greater percentage of the world population.

Magnetic resonance imaging of the human brain requires the patient to remain motionless for extended periods of time to avoid artefacts and reduced image quality. However, motion of the head is a common problem in clinical and empirical applications of this form of technology. Typically, a patient is required to lie flat in the supine position and stay still for long periods of time within the magnet bore of the MRI machine. From a patient's perspective, the enclosed space inside the bore and the extended time period that the patient is asked to remain still can cause enormous anxiety prior to and during the MRI scan. A feeling of lack of control and a feeling of discomfort or vulnerability when in the supine position have also been identified as negative factors in the patient experience.

The applicant considers that the development of a seated, head-only imaging system dedicated to brain imaging, which is one of the most common applications for MRI scans, would offer a less restrictive scanning procedure. It could also contribute to the patient feeling a greater level of control due to being in an upright position and being able to look outside the magnet using a small window. Seated MRI scanners also take up less horizontal space and therefore offer space saving cost benefits to hospitals and clinics that house MRI scanners. However, preferably the patient should move their head less than 1 mm during image acquisition. In a seated position, the head is less stable than when the patient is lying supine. Additional support must therefore be supplied to the patient's head in a seated MRI system to reduce head movement to acceptable levels.

Several commercial devices used as part of conventional MRI systems aim to provide a stable head position during the scanning process. Such devices include the use of polyurethane foam pads, thermoplastic nets moulded to the face of the patient, support pillows and medical tape to tape the patient's head in position. The rate of acceptance of these support devices can vary among patients, and some of the head support designs are not compatible with high-channel count radio frequency (RF) receive coils. Furthermore, known head stabilisation systems for MRI are designed to restrict movement about the longitudinal axis, rather than about all axes, as is required without the support of a patient bed. Finally, the known head stabilisation systems are designed to be used by a patient placed in a supine position prior to use, and therefore may not be suitable for seated MRI scanning.

Inflatable head supports may be used for seated MRI scanning, but known head supports must be inflated before the dedicated head multi-channel receive coil is placed over the patient's head. This can make it difficult for a clinician to properly position the coil over the bulky head support. It can also be uncomfortable for the patient as the head support and coil interact and press against each other.

It would be useful to provide an inflatable head support for use by a patient during seated MRI scanning that goes at least some way towards overcoming the disadvantages of the prior art or that at least provides the public with a useful alternative to known head supports.

### SUMMARY OF THE INVENTION

In a first aspect, the present disclosure relates to a stabilising head support for use by a patient during MRI scanning, the head support comprising a shell and an inflatable bladder comprising a gas inlet and a gas outlet and being located at least partially within the shell. The bladder forms a generally annular structure comprising a front portion, opposing side portions and a rear portion to contact the front, sides, and rear of the patient's head respectively. A portion of the bladder extends below a rear portion of the shell to contact the upper back of the patient.

In some forms, the rigid shell comprises a front portion, opposing side portions and a rear portion and wherein the rear portion is formed in two parts separated by an opening that extends from a bottom edge to a top edge of the rear portion.

Optionally, the rigid shell and bladder each comprise a top portion having an opening for receiving at least a portion of a crown of the patient's head.

In some forms, the gas inlet is located at the rear portion of the bladder. Optionally, the gas inlet is located at the front portion of the bladder.

In some forms, the rear portion of the bladder is between about 200 mm and about 400 mm long. Preferably, the rear portion of the bladder is about 200 mm long.

In some forms, the bladder comprises thermoplastic polyurethane coated nylon.

Optionally, the inflatable bladder comprises two or more independently inflatable members that are connected together.

In some forms, the bladder comprises a front portion that is separately inflatable to the remainder of the bladder.

Optionally, the front portion is adapted to inflate before the remainder of the bladder.

In some forms, the shell comprises an RF coil.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

Directional terminology used in the following description is for ease of description and reference only, it is not intended to be limiting. For example, the terms ‘front’, ‘rear’, ‘upper’, ‘lower’, ‘top’, ‘bottom’ and other related terms refer to the location of a part or portion of the article being described, when the article is in use.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

### BRIEF DESCRIPTION OF THE DRAWINGS

Embodiments will now be described by way of example only and with reference to the accompanying drawings in which:

FIG. 1 is an isometric front view from above of one form of shell to be used with a stabilising head support of the present invention;

FIG. 2a is an isometric rear view from above of the shell of FIG. 1 where possible locations for fasteners are visible to attach the shell to a compressible inner/bladder;

FIG. 2b is an isometric rear view from above of another form shell that uses a tacky interior surface to attach the shell to a compressible inner/bladder;

FIG. 3a is an isometric rear view of one form of compressible inner/bladder in a deflated state;

FIG. 3b is an isometric rear view of the compressible inner/bladder of FIG. 3a in an inflated state and having a gas inlet and outlet at a lower region of a rear portion of the bladder;

FIG. 4 is a front view of another form of compressible inner/bladder, having a gas inlet and outlet at a front portion of the bladder at the forehead region;

FIG. 5 is an isometric rear view of yet another form of compressible inner/bladder formed in two portions, a first portion and a second portion, each of the first and second portions having a gas inlet and outlet located at a lower region of a rear portion of the respective bladder;

FIG. 6 is yet another form of compressible inner/bladder, having supporting side portions adapted to at least partially wrap around the sides of a patient's face;

FIG. 7 is a front view of the bladder of FIG. 6 when worn by a patient;

FIG. 8 is an isometric rear view of one form of head support according to the invention that includes one form of deflated bladder located within one form of shell;

FIG. 9 is a front view of the head support of FIG. 8;

FIG. 10 is a front view of the head support of FIG. 8 when worn by a patient;

FIG. 11 is a side view of the head support of FIG. 8 when worn by a patient and showing a rear portion of the patient's head exposed through an opening in the upper rear of the head support;

FIG. 12 is a rear view of the head support of FIG. 8 when worn by a patient, showing a portion of the bladder extending below the shell;

FIG. 13 is an exploded view of an RF coil, a shell, and a compressible inner/bladder according to form of the invention, the compressible bladder being configured to nest within the shell and the RF coil being configured to fit over the head support formed by the shell and bladder combination;

FIG. 14 is an isometric view from below showing one form of shell of the invention located within an RF coil;

FIG. 15 is an isometric view from the front showing one form of head support according to the invention located within an RF coil, in which a rear portion of the bladder of the head support extends below the RF coil;

FIG. 16 is an isometric view of one form of MRI machine for seated MRI scanning; and

FIG. 17 is a schematic view showing the x, y, and z axes in relation to a patient's head.

### DETAILED DESCRIPTION

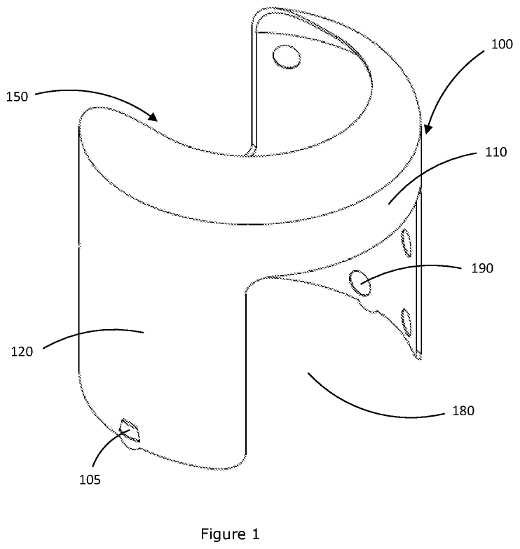

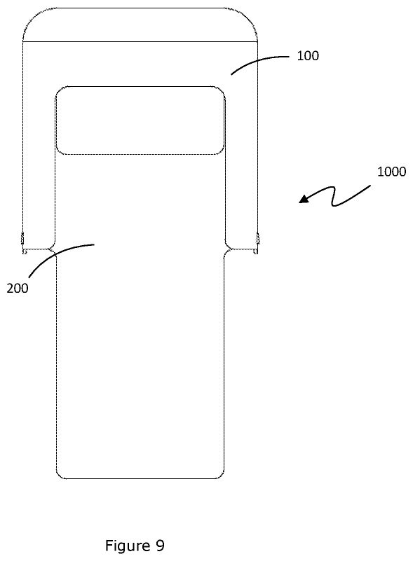

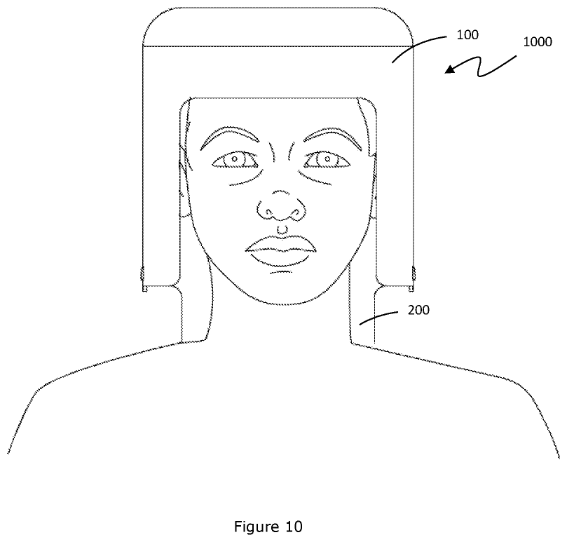

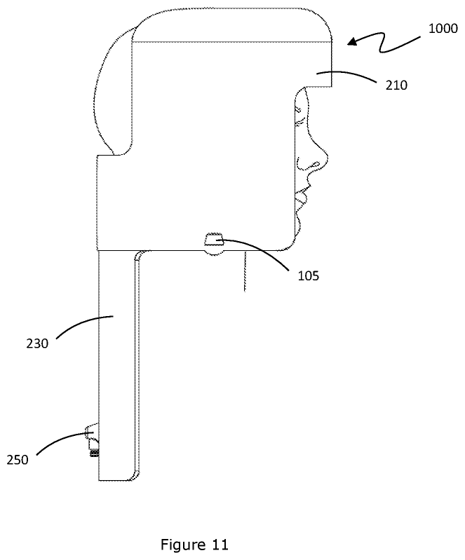

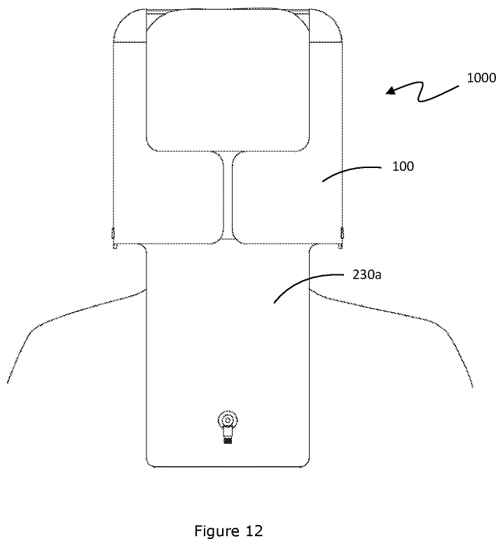

Various embodiments of the invention and methods of manufacture will now be described with reference to FIGS. 1 to 17. In these figures, like reference numbers are used to indicate like features.

Referring firstly to the embodiments shown in FIGS. 1 to 15, the present invention provides a stabilising head support 1000 adapted to fit over/around the head of a patient during imaging, and preferably during seated MRI scanning/imaging. The head support 1000 is adapted to support the patient's head from the front, sides and rear in order to help stabilise the position of the patient's head in the x, y and z directions over long periods of time, such as for more than 10 minutes or more than 20 minutes.

The head support 1000 is adapted to be located within an annular radiofrequency (RF) coil 2000 that generates a magnetic field for use in image scanning, such as MRI. Radiofrequency cons are the receivers, and sometimes also the transmitters, of RF signals in equipment used in MRI. In use, the annular RF coil 2000 surrounds the head support 1000 such that the head support is located within the annulus or bore of the coil 2000. A patient is then seated beneath the head support 1000 and coil 2000 and the coil and head support together are then positioned to substantially surround the patient's head by lowering the coil and head support over the patient's head or by raising the seating platform on which the patient is sitting until the patient's head is received within the head support 1000 and RF coil 2000. Once in position, the patient's head is located within the bore of the coil 2000.

The head support 1000 comprises a hard, rigid, or semi-rigid outer shell 100 and a compressible inner portion/bladder 200.

The bladder is adapted to inflate by receiving gas through a gas inlet of the bladder, at which point the compressibility of the bladder is reduced so that the bladder is sufficiently firm to support the position of a patient's head within the head support and to steady the head during imaging. When inflated, an inner surface of the bladder presses against the patient's head to exert a stabilising pressure against the head, preferably from all sides (front, rear, left and right sides), and an outer surface of the bladder presses against an inner surface of the outer shell. In this arrangement the bladder stabilises and substantially immobilises the head within the shell.

The bladder is also adapted to deflate when the patient is to be removed from RF coil or when the head support is not in use. When the head support is to be removed from a patient's head, the bladder is first deflated by releasing gas through a gas outlet of the bladder. Upon deflation, the bladder becomes flexible and its overall dimensions are reduced so that the inner surface of the bladder no longer exerts a stabilising pressure against the patient's head. The patient can then be removed from the head support and RF coil.

In some forms, the bladder may comprise a dedicated gas inlet and a dedicated gas outlet. In preferred forms however, the bladder comprises a single opening that comprises both the gas inlet and gas outlet.

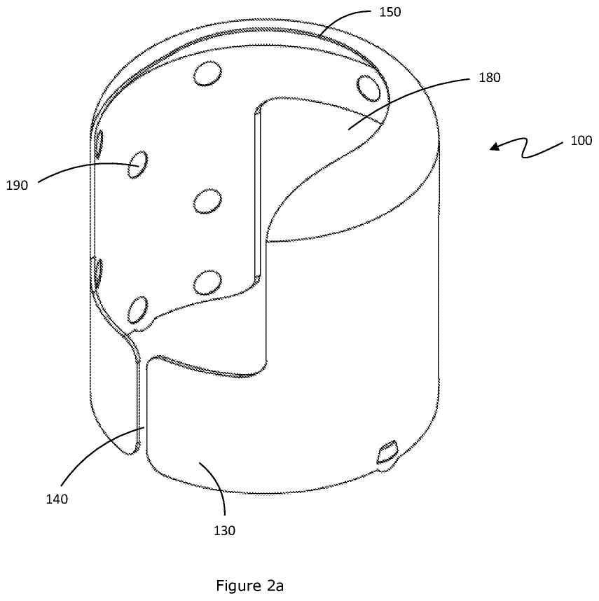

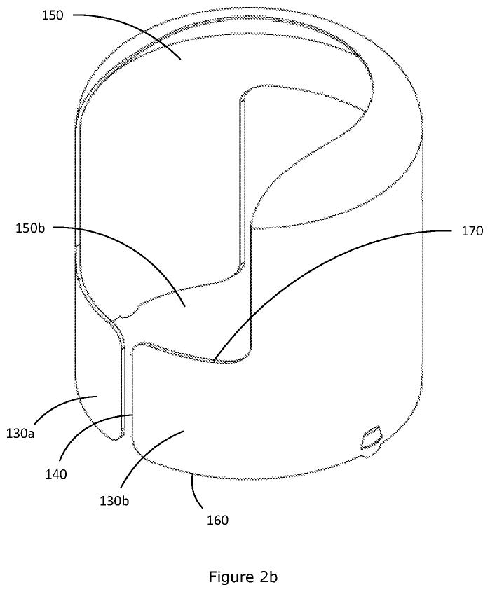

As shown in FIGS. 1 to 6, the head support 1000 comprises an outer shell 100 made from a hard, non-magnetic, non-electrically conductive, hospital grade material, as shown in FIGS. 1, 2a, and 2b. The shell 100 acts as a carrier for the compressible inner portion/bladder 200 and holds the bladder in place as a patient's head enters and exits the head support. In some embodiments, the shell may be flexible to some extent to allow removal of the complete head support assembly (shell and bladder) from within the RF coil for cleaning or maintenance.

In some forms the shell comprises a plastic material suitable for injection moulding such as polypropylene or polycarbonate. In other forms, the shell comprises a plastic suitable for 3D printing such as acrylonitrile butadiene styrene (ABS) or photopolymers suitable for use with a stereolithography process. In other forms, the shell comprises a composite material such as fiberglass.

The outer shell 100 is adapted to substantially surround the circumference of a patient's head, including at least a portion of each of the front (forehead), sides and rear of the head. In this regard, the shell 100 comprises a front portion 110, opposing side portions 120 and a rear portion 130 adapted to be located respectively adjacent to the front/forehead, sides, and rear of a person's head during use. In preferred forms, the front portion 110 extends across the forehead region of the patient and generally leaves the rest of the patient's face exposed. The side portions 120 may extend partially or fully along each side of the patient's head. The rear portion 130 may extend partially or fully across the rear of the head. Where the rear portion extends only partially across the rear of the head, the rear portion may extend across an upper portion or a lower portion or a central portion of the rear of the patient's head.

In some forms, as shown in FIG. 2b, the rear portion 130 of the outer shell 100 comprises two parts/portions 130a, 130b, separated by an opening or split 140 that extends from a lower edge 160 of the rear portion 130 to an upper edge 170 of the rear portion 130, as shown in FIGS. 2a and 2b. The split 140 allows the two portions 130a, 130b of the rear portion to flex slightly to assist with removing the shell from the RF coil. In effect, the split 14 allows the two portions 130a, 130b of the rear portion to flex outwardly, away from an interior of the shell, and to flex inwardly toward the interior of the shell. By flexing the two rear portions 130a,130b inwardly or by flexing the shell such that the two rear portions 130a, 130b move toward each other, the shell may be readily separated from the coil 2000 and removed from the coil. In some forms, the split 140 may also act as a guide to guide the position of the shell 100 in relation to the RF coil or MRI magnet.

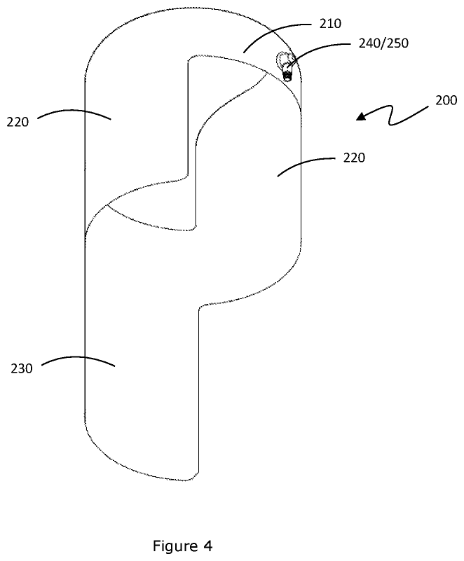

In some forms, an opening 150 is provided at the top of the shell 100 for receiving the crown of the patient's head. In some forms, the top section opening 150 may extend rearwardly to include an opening 150b at the rear of the shell 100 so that a portion of the crown/top of the patient's head and a portion of the rear of the patient's head are exposed, as shown in FIG. 4. The top section opening 150 allows the top of the head to touch a datum surface at the top of the RF coil when the patient's head is located within the coil.

The shell 100 comprises a face opening 180 at the front portion. The face opening 180 is located so that at least a portion of a patient's face is exposed by the opening. Preferably, the opening is sized and shaped to expose at least the patient's eyes, nose, and mouth. In some forms, the face opening 180 comprises a cut-away region extending from a lower edge 160 of the shell 100 and toward the top of the shell 100 to define a generally arched face opening 180. In this form, the front portion 110 of the shell 100 extends across the top of the arched face opening 180 to generally correspond with a patient's forehead, leaving exposed the area of the patient's face below. In this form, the shell and head support may resemble an open face helmet in its general shape. In other forms, the shell may include a chin portion that extends across the chin region of the patient's face from one side of the shell to the other. In this arrangement, the face opening is located between the forehead portion and chin portion of the shell and the head support may resemble a closed face helmet in its general shape.

In some forms, the shell 100 may be adapted to detachably attach to the RF coil 2000. Any suitable attachment system may be used to attach the shell to the RF coil. For example, one or more fasteners may be used, or the outer surface of the shell and inner surface of the coil may be configured to attach to each other In some forms, fasteners in the form of clips 105 are provided on the outer surface of the shell, near the lower edge of the shell 100, for clipping onto the RF coil 2000. In another form, the shell and coil include structural features that allow the shell and coil to be located together such that the shell nests within the coil. For example, a flange or ledge having an upwardly facing mounting surface may project from the outer surface of the shell, preferably near the lower edge of the shell, such that the RF coil may rest on the contact surface. In another form, an inner surface of the coil may comprise a recess or channel and an outer surface of the shell may comprise a projection configured to be received within the recess or channel to mate/engage the shell and coil together. Preferably, the connection is moveable or flexible to allow for easy disassembly of the shell and coil. Other attachment systems may alternatively be used to support the RF coil in position in relation to the head support 1000 as would be appreciated by a person skilled in the art. It is important that any attachment system allows the head support 1000 to remain in a stable position within the RF coil 2000 and to be removed from the coil when required.

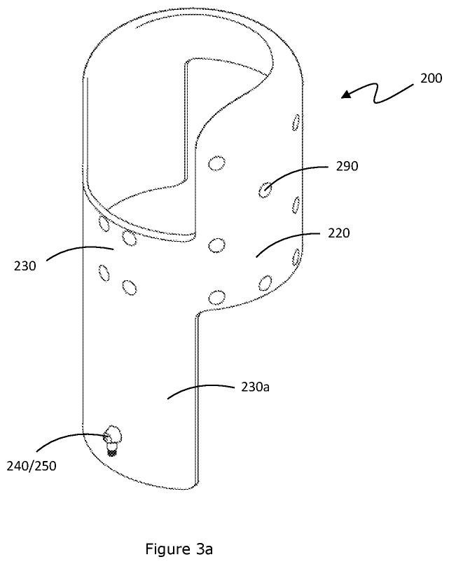

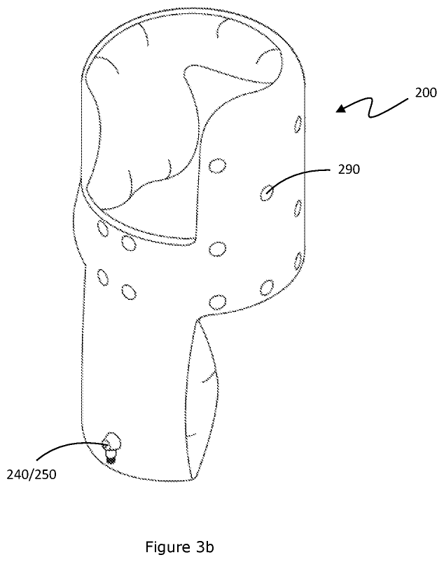

FIGS. 3a and 3b to 7 show various forms of compressible inner portions/bladders 200 that may be used with the head support 1000 of the invention. The bladder 200 is adapted to deflate, as shown in FIG. 3a, and inflate, as shown in FIG. 3b. The bladder 200 forms a generally annular structure comprising a front portion 210, opposing side portions 220 and a rear portion 230. The inflatable bladder 200 is locatable within the rigid or semi-rigid shell so that the front portion 210, side portions 220 and rear portion 230 of the bladder 200 are respectively located adjacent inner surfaces of the front portion 110, side portions 120 and rear portion 130 of the shell 100. The front portion, side portions and rear portion of the bladder are arranged respectively to contact the front, sides, and rear of the patient's head in use.

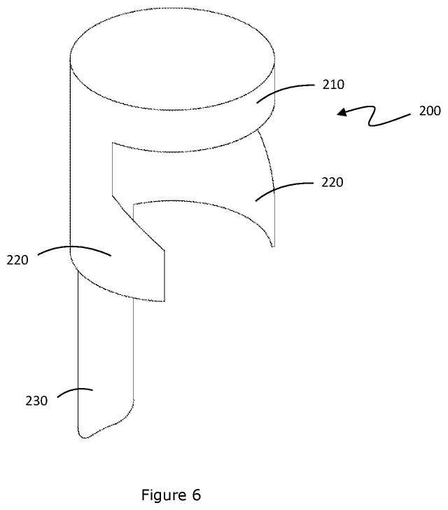

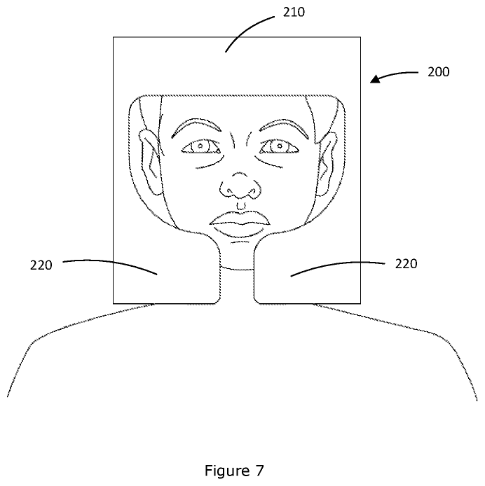

In some forms, as shown in FIGS. 6 and 7, side portions 220 of the bladder 200 may at least partially wrap around the patient's face, by extending along the cheeks or jaw region and toward the patient's nose. In some forms, the bladder 200 may be configured to at least partially support the bottom of the patient's jaw by extending along the jawline.

The bladder 200 comprises a face opening 240 that exposes at least a portion of the patient's face, including the patient's eyes, nose, and mouth when the head support is worn by the patient. In some forms, the face opening 240 extends upwardly from the lower edge of the bladder 200 to form a generally arched opening. In some forms, the front portion of the bladder is adapted to contact the forehead of a patient wearing the head support 1000, leaving the lower portion of the face exposed and distanced from the inner surface of the shell to enable airflow. In another form, the bladder comprises a chin portion that extends across the patient's chin during use, such that the face opening is located between the forehead portion and chin portion of the bladder.

The front portion of the shell 100 and bladder 200 together form a front portion of the head support 1000 that is, in use, located adjacent to and supports the front of a patient's head. The side portions of the shell and bladder together form opposing side portions of the head support that are located adjacent to and support the sides of the patient's head in use. Similarly, the rear portions of the shell and bladder together form a rear portion of the head support 1000 that is, in use, located adjacent to and supports the rear of the patient's head.

In some forms, the rear portion 230 of the bladder 200 comprises an extension 230a that extends below the rear portion of the rigid shell 100. Preferably, the rear extension 230a comprises a gas inlet and gas outlet to allow air to be injected into and released from the bladder at the rear. The gas inlet and gas outlet may each be independent of each other, but preferably form a single conduit to the interior of the bladder.

The rear portion extension 230a may also provide additional support and therefore stability to the patient's neck. The rear portion extension 230a of the bladder rests against the patient's neck and upper back and has been found to be useful in assisting the stabilisation of a patient's head during seated image scanning.

The inflatable bladder 200 forms a generally annular structure that surrounds the circumference of a patient's head when worn by the patient. The top section of the bladder preferably comprises an opening for receiving at least a portion of the top/crown of the patient's head so that at least a portion of the top/crown is exposed. In some forms, the top section opening extends toward the rear of the bladder 200, as shown in FIG. 4, so that both a portion of the top/crown of the patient's head and a portion of the rear of the patient's head are exposed.

In some forms, the shell 100 is detachably attachable to the bladder 200 via any suitable attachment system so that the bladder can be readily attached to and removed from the shell. In some forms, the attachment system comprises one or more fasteners 190. For example, one or more inner surfaces of the shell 100 may comprise fasteners 190 adapted to detachably attach to the bladder 200 to the shell. In some forms, complimentary hook, and loop fasteners 190, 290 may be located on the shell 100 and bladder 200 to detachably attach the shell 100 and bladder 200 together, as shown in FIGS. 1, 2a, 3a and 3b. In another form, one or more inner surfaces of the shell 100 may be coated with or comprise a tacky substance to loosely adhere with the bladder 200 and/or one or more outer surfaces of the bladder may be coated with or comprise a tacky substance to loosely adhere with the shell 100, as shown in FIG. 2b. In other forms, the shell 100 and bladder 200 may comprise complimentary domes, clips, or any other suitable fasteners or attachment system to detachably attach the shell 100 and bladder 200. The detachable nature of the bladder provides the ability to remove the bladder 200 from the shell 100 so that the bladder and shell can be cleaned, maintained, or replaced.

The bladder 200 may comprise any suitable flexible, non-magnetic, non-electrically conductive hospital grade material that allows the bladder to be inflated and deflated. Because the bladder 200 will be filled with a gas, the bladder needs to be made from or coated with a material that is impervious to gas. In preferred forms, the bladder is adapted to be filled with air. In some forms, the bladder comprises thermoplastic polyurethane coated nylon.

Preferably, the bladder material is also heat sealable to join edges of the bladder together in a sealed arrangement. In some forms, the bladder 200 may comprise heat welded seams, ultrasound welded seams or any other form of sealed seams to retain gas within the bladder.

Preferably, the bladder is shaped to at least partially mould around a patient's head when inflated.

In some forms, the bladder comprises thermoplastic polyurethane (TPU) coated nylon, polyvinyl chloride (PVC) coated nylon, vinyl, or oxford cloth at around 170 g/m².

The inflatable bladder 200 comprises at least one gas passage/inlet 240 through which gas can enter the bladder to inflate the bladder. The bladder 200 also comprises at least one gas passage/outlet 250 through which the gas can exit the bladder to deflate the bladder. In some forms, the inlet may also act as the outlet. In other forms, the bladder 200 may comprise one or more gas outlets that are separate/distinct from the one or more gas inlets.

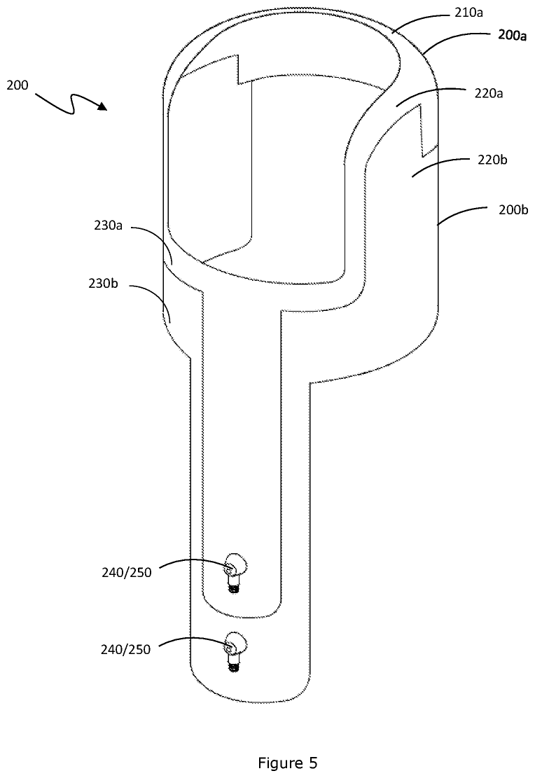

The inlet(s) 240 and outlet(s) 250 may be located at any suitable location on the bladder 200. In a preferred embodiment, the bladder 200 comprises an inlet and/or outlet 240, 250 located on the rear portion 230 of the bladder, such as on the extension portion 230a of the rear portion of the bladder and preferably on a lower region of the extension portion 230a, as shown in FIGS. 3a, 3b and 5. In this arrangement, the inlet 240 and/or outlet 250 is/are easily accessible even when a patient is wearing the head support 1000. The inlet/outlet 240/250 shown in FIG. 3a acts as both an inlet and an outlet. In one form, as shown in FIG. 4, the bladder 200 comprises an inlet 240 and/or outlet 250 at the front portion of the bladder. In another form, the bladder comprises an inlet and/or outlet at one of the side portions of the bladder.

In some forms, the rear portion of the bladder is about 200 mm long and is preferably between about 200 mm and about 400 mm long.

In some forms, the bladder 200 may consist of a single inflatable section, as shown in FIG. 3a. In other forms, as shown in FIGS. 4 and 5, the bladder 200 may comprise at least two independently inflatable members that are connected together. Each of the inflatable portions comprises a gas inlet and/or outlet, as shown in FIG. 5. The inflatable members may be attached together to form a surround or wrap around the circumference of a patient's head. For example, as shown in FIG. 5, the two inflatable members of the bladder may comprise a first member 200a and a second member 200b that are attached together, preferably along side edges of the inflatable members. The first member 200a may comprise the front portion 210a of the bladder 200 that extends across the forehead of a patient in use. The first member 200a comprises a front portion 210a, a rear portion 230a and a pair of side portions 220a spanning between the front and rear portions 210a, 230a to form a generally annular arrangement. The front portion 210a is generally connected to the side portions 220a that wrap around the sides of the patient's head and that connect to the rear portion 230a located at the rear of the patient's head during use. The first member 200a may comprise a first gas passage inlet/outlet, which may be located at any suitable location, such as at the front portion 210a of the bladder, in front of the patient's forehead, as shown in FIG. 4, or at the rear 230a of the bladder, such as on a rear extension portion of the first member, as shown in FIG. 5. In some forms, the inlet 240 may be located at the front portion 210a and the outlet 250 may be located at the rear portion 230 or vice versa.

The second member 200b may be attached to the first member 200a, by any suitable attachment system, such as by welding, heat sealing, one or more fasteners, hook and loop fasteners, or clips for example. Alternatively, the first and second members 200a, 200b may be integrally formed as a single part and then separated into two members by a gas impermeable seal that defines the join between the first and second members. The second member 200b preferably comprises at least a pair of generally opposing side portions 220b and a rear portion 230b that extends between the side portions 220b. In some forms, the second member 200b also provides a front portion 210b that extends across a portion of the patient's forehead and extends between the side portions 220b generally opposite to the rear portion 230b.

Where the bladder comprises two inflatable members, the first member 200a is preferably inflated before the second member 200b and is preferably deflated after the second member 200b. By inflating the first member 200a first, the front portion 210 of the bladder is inflated before the majority of the sides 220 and rear 230 of the bladder are inflated. By inflating the front portion 210 before the rear portion 230 or a majority of the rear portion, it is possible to use the inflated front portion 210 to push against an inner surface of the RF coil to push the patient's face away from the RF coil before inflating the remainder of the bladder 200. This helps prevent the patient's face and nose especially from being pushed against the RF coil as the bladder inflates.

In another form, the head support may comprise a bladder comprising a front portion connected to a pair of side portions and a generally opposing rear portion but that is separately inflatable and deflatable from the rear and side portions. In this arrangement, the front portion includes a gas inlet and gas outlet and the rear and side portions together comprise a gas inlet and gas outlet that is separate to that of the front portion. A gas impermeable seal is provided between the front portion and the side portions. In this form, the front portion 210 of the bladder is separately inflatable from the remainder of the bladder to allow the front portion to be inflated first and optionally deflated last to distance a patient's face from the inner surface of the RF coil during use.

In some forms, the shell 200 forms the RF coil, such that the bladder nests directly within the coil and the outer surface of the bladder contacts the inner surface of the coil.

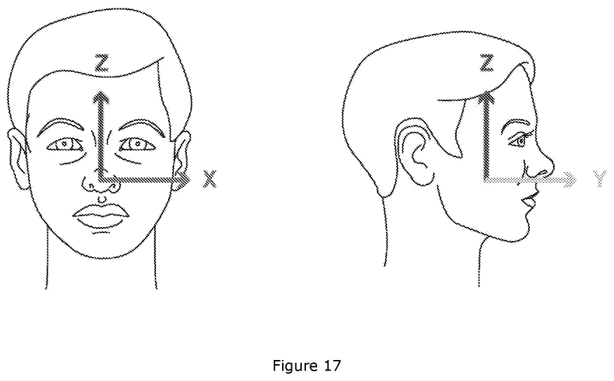

The inflatable bladder 200 provides a compressible surface around the patient's head to provide the patient with a level of comfort while applying stabilising pressure to the patient's head, from at least three sides (such as from the front and sides of the bladder), and preferably from all sides. This is achieved by placing a patient's head within the bladder within the shell and/or RF coil. The bladder is then inflated such that the outer surface of the bladder presses against the inner surface of the shell or coil, and the inner surface of the bladder presses against the patient's head. The bladder is therefore held under compression and applies pressure around the patient's head to hold the patient's head in a steady position within the shell or coil and to generally preclude movement of the patient's head along the x, y and z axes, as identified in FIG. 17. The bladder 200 helps to locate/position the patient's head correctly and at a consistent position within the shell 100 and therefore within the RF coil/magnet bore over time as imaging occurs.

In some forms, the bladder 200 has a size of approximately 800 mm long/around×400 mm wide when deflated.

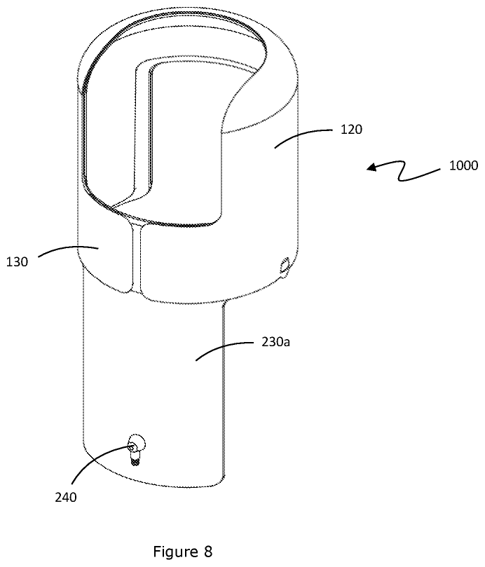

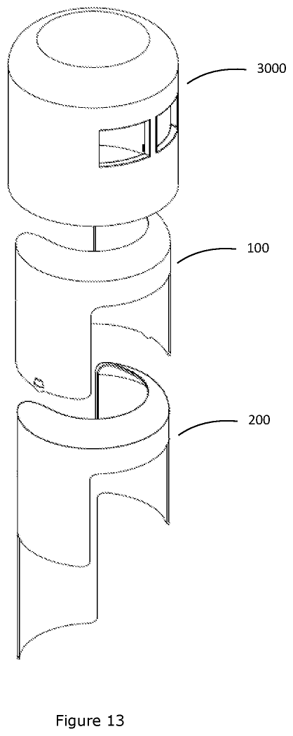

FIGS. 8 to 12 show one form of bladder 200 positioned inside one form of shell 100 according to the invention. The bladder 200 and shell 100 together form the head support 1000 that is located within the RF coil 2000 of an MRI machine, as indicated in FIG. 13.

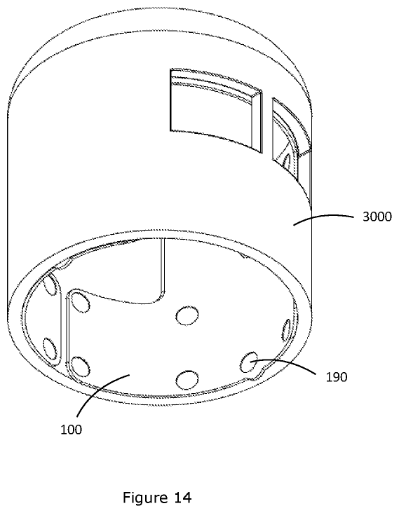

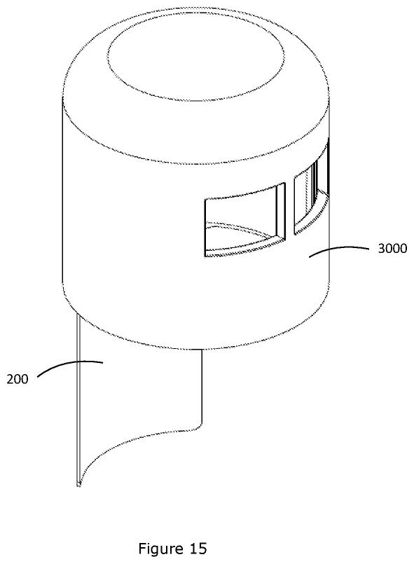

To use the head support 1000 for seated MRI scanning, the shell 100 is placed within an RF coil 2000 of an MRI machine and held in position by any suitable attachment system or mounting system. For example, a mounting system, clips 105 or other fasteners may engage with the shell and the RF coil to hold the shell 100 in place relative to the RF coil, as shown in FIG. 14. The bladder 200 is then inserted within the interior of the shell 100 and is attached to the shell 100 to form the head support 1000 within the RF coil 2000, as shown in FIG. 15. The bladder 200 is prepared for inflation by attaching the inlet 240 to a gas supply, such as a compressed air supply, with a valve to control gas flow into the bladder.

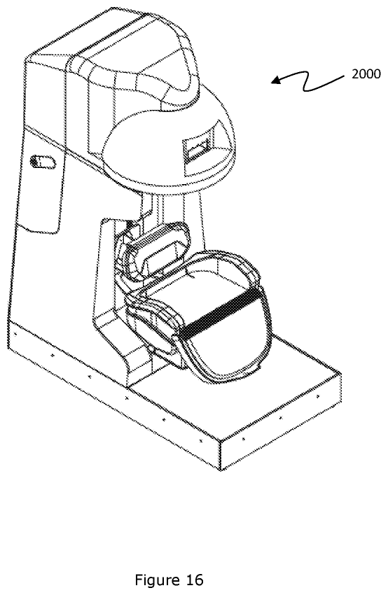

A patient is seated on a chair of the MRI machine 2000, as shown in FIG. 16. The chair is raised to position the patient's head within the head support 1000 so that the inflatable bladder 200 surrounds the patient's head. In some forms, the chair is raised until the patient's head activates a switch at the top of the RF coil 2000 to stop the chair from moving. In preferred forms, the switch is a button. In other forms, the patient or clinician may stop the chair from being raised further via a user interface of the MRI machine. In yet other forms, the RF coil and head support may be lowered over the patient's head.

Once the patient is in position so that his or her head is correctly positioned within the head support 1000 and coil 2000, the bladder 200 is inflated, such as by the patient or a clinician. In some forms, the front/forehead portion of the bladder 200 is inflated first and then the remainder of the bladder is inflated. The bladder 200 is inflated to the extent that the patient remains comfortable with the level of inflation and the patient's head is held in a stable position within the head support 1000 and coil 2000 from all sides so that the patient's head is generally prevented from substantially moving in the x, y and z axes.

Scanning of the patient's head can then take place as the head support 1000 helps the patient to keep his or head still during the scanning. Once the scanning is complete, the bladder 200 is deflated, typically by the patient or a clinician, and then the patient is removed from the RF coil, such as by lowering the chair or raising the coil 2000 to extract the patient from the RF coil 2000.

The inflatable head support 1000 may be used to provide a semi-compressible support between a patient's head and the magnetic RF coil 2000 during MRI scanning. The inflatable head support 1000 is configured to comfortably hold the patient's head in position within the RF coil 2000 for extended periods of time, such as between about 0 and about 20 minutes, including between about 10 minutes and about 20 minutes.

The head support 1000 has been shown to help stabilise and locate the patient's head within an MRI machine 2000. Tests show that the head support 1000 has been shown to minimise head movement in each of the x, y, and z orientations for up to 20 minutes, the x, y, and z orientations being indicated in FIG. 17.

Tests were carried out to assess the effectiveness of the head support 1000. Test data set out in the tables below show that the head support was useful to restrict movement of a patient's head in each of the x, y, and z axes.

TABLE 1
Baseline recording with no head support of patient over 120 seconds.

| | Baseline recording | | | | | |
|---|---|---|---|---|---|---|
| Length | 120 seconds | | | | | |
| Details | No head support, no arm movement | | | | | |
| | Rotation x | Rotation y | Rotation z | Movement x | Movement y | Movement z |
| | (in degree) | (in degree) | (in degree) | (in mm) | (in mm) | (in mm) |
| P01 | −0.008 to | −0.006 to | −0.025 to | −0.6 to 2.7 | −1.9 to 0.6 | −0.5 to 1.6 |
| | 0.022 | 0.001 | 0.008 | | | |
| P02 | −0.005 to | −0.023 to | −0.040 to | −0.2 to 1.7 | −1.2 to 1.5 | 0.8 to 0.6 |
| | 0.021 | 0.008 | 0.024 | | | |
| P03 | −0.041 to | −0.267 to | 0.97575 to | −0.8 to 0.1 | −1 to 0.2 | −0.3 to 0.1 |
| | −0.038 | −0.254 | 0.96 | | | |
| P04 | −0.021 to | 0.005 to | 0.003 to | −0.2 to 1.8 | −0.6 to 1 | −2.8 to 0.3 |
| | −0.003 | 0.008 | 0.014 | | | |
| P05 | 0.132 to | 0.846 to | 0.288 to | −2.75 to 0.3 | −1 to 0.7 | −0.3 to 1.5 |
| | 0.142 | 0.8495 | 0.297 | | | |

TABLE 2
Test with head support of patient over 120 seconds.

| Length | 120 seconds | | | | | |
|---|---|---|---|---|---|---|
| Details | Head support, no arm movement | | | | | |
| | Rotation x | Rotation z | Rotation y | Movement x | Movement y | Movement z |
| | (in degree) | (in degree) | (in degree) | (in mm) | (in mm) | (in mm) |
| P01 | 0.010 to | −0.006 to | 0.0002 to | −1.3 to 0.5 | −1.5 to 0.2 | 0.6 to 0.4 |
| | 0.028 | 0.002 | 0.019 | | | |
| P02 | −0.008 to | 0 to | −0.002 to | −0.2 to 0.1 | 0 to 1.7 | −0.1 to 0.5 |
| | −0.002 | 0.004 | 0.008 | | | |
| P03 | −0.006 to | −0.0018 to | −0.0012 to | −1.8 to 0 1 | −0.4 to 0.8 | −0.5 to 0.1 |
| | 0.0013 | −0.014 | 0.007 | | | |
| P04 | −0.02 to | 0.015 to | −0.001 to | −1 to 0.6 | −0.5 to 0.7 | −1.1 to −0.1 |
| | 0.005 | 0.0185 | 0.008 | | | |
| P05 | −0.251 to | −0.077 to | −0.961 to | 0.6 to 0.2 | −2.3 to 0.2 | −0.6 to 0.5 |
| | 0.251 | 0.060 | 0.362 | | | |

The invention may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Preferred embodiments of the invention have been described by way of example only and modifications may be made thereto without departing from the scope of the invention

1. A stabilising head support for use by a patient during MRI scanning, the head support comprising a shell and an inflatable bladder comprising a gas inlet and a gas outlet and being located at least partially within the shell, wherein the bladder forms a generally annular structure comprising a front portion, opposing side portions and a rear portion to contact the front, sides and rear of the patient's head respectively, and wherein a portion of the bladder extends below a rear portion of the shell to contact the upper back of the patient.

2. The stabilising head support of claim 1, wherein the rigid shell comprises a front portion, opposing side portions and a rear portion and wherein the rear portion is formed in two parts separated by an opening that extends from a bottom edge to a top edge of the rear portion.

3. The stabilising head support of claim 1, wherein the rigid shell and bladder each comprise a top portion having an opening for receiving at least a portion of a crown of the patient's head.

4. The stabilising head support of claim 1, wherein the gas inlet is located at the rear portion of the bladder.

5. The stabilising head support of any one of the preceding claim 1, wherein the gas inlet is located at the front portion of the bladder.

6. The stabilising head support of claim 1, wherein the rear portion of the bladder is between about 200 mm and about 400 mm long.

7. The stabilising head support of claim 1, wherein the rear portion of the bladder is about 200 mm long.

8. The stabilising head support of claim 1, wherein the bladder comprises thermoplastic polyurethane coated nylon.

9. The stabilising head support of claim 1, wherein the inflatable bladder comprises two or more independently inflatable members that are connected together.

10. The stabilising head support of claim 1, wherein the bladder comprises a front portion that is separately inflatable to the remainder of the bladder.

11. The stabilising head support of claim 10, wherein the front portion is adapted to inflate before the remainder of the bladder.

12. The stabilising head support of claim 1, wherein the shell comprises an RF coil.
